(19) United States
(12) Patent Application Publication
(10) Pub. No.: US 2024/0374785 A1
(43) Pub. Date: Nov. 14, 2024

(54) Gelation of Highly Entangled Hydrophobic Macromolecular Fluid for Ultra-Strong Underwater In-Situ Fast Adhesion to Artery, Lung, Bone and Skin Tissues

(71) Applicant: University of Manitoba, Winnipeg (CA)

(72) Inventor: Malcolm Xing, WInnipeg (CA)

(21) Appl. No.: 18/690,078

(22) PCT Filed: Oct. 26, 2022
(86) PCT No.: PCT/CA2022/051582

§ 371 Date: Mar. 7, 2024

Related U.S. Application Data
(60) Provisional application No. 63/273,533, filed on Oct. 29, 2021.

(51) Int. Cl. A61L 26/00; A61L 24/00; A61L 24/04

(52) U.S. Cl. CPC A61L 26/0019; A61L 24/0026; A61L 24/0042; A61L 24/046; A61L 26/0076; A61L 26/009; A61L 2400/04

(57) ABSTRACT

Building strong underwater bioadhesion is important for several applications but proved to be an extremely challenging task. In a wet/underwater environment, the water boundary layer hinders interfacial adhesion, which is further undermined by water-induced swelling in bioadhesives. In contrast to current hydrophilic bioadhesives, we here propose an underwater and in-situ applicable hydrophobic adhesive (UIHA). We report polydimethylsiloxane (PDMS) tangled with macromolecular silicone fluid as a dissipation phase to provide strong interface adhesion strength. The silicone fluid repels the surface boundary water layer, overcomes capillary break up in water, and rapidly gelates with PDMS, while a small amount of silane (<0.2%) can bridge the hydrophobic adhesive and tissue substrates to an exceptional underwater adhesive strength. UIHA presents in-situ and instant adhesive performances when tested on artery, lung, bone, and skin tissues.

### PRIOR APPLICATION INFORMATION

The instant application claims the benefit of U.S. Provisional Patent Application Ser. No. 63/273,533, filed Oct. 29, 2021, entitled “GELATION OF HIGHLY ENTANGLED HYDROPHOBIC MACROMOLECULAR FLUID FOR ULTRA-STRONG UNDERWATER IN-SITU FAST ADHESION TO ARTERY, LUNG, BONE AND SKIN TISSUES”, which is incorporated herein by reference for all purposes.

### BACKGROUND OF THE INVENTION

Wet adhesives are applicable in all spectrums of tissue trauma where instant wound closure and hemostasis are needed, especially when time is critical in life rescue [1-7]. Instant and strong wet/underwater adhesion performance is also required for wound closure in the scenarios of blood or body fluid exposure. However, adhesion to wet substrates or underwater surfaces is still a challenge, as water molecules in the boundary layer of the interface impede the direct contact between adhesives and substrates [2, 8-11]. Inspired by marine mussels, sandcastle worms, tree frogs, clingfish, and geckos [12-15], several approaches were proposed to overcome surface water, including coacervation of polyelectrolytes, catecholic bonding, dry hydrogel tapes absorption, or repelling water through micropattern [1, 9, 14-23].

Many of the recently reported bioadhesives for wet/underwater applications are hydrophilic polymers [1, 2, 9, 23]. Since water is absorbed to the interface because of the bioadhesive hydrophilicity, adhesion either fails altogether or—if it does not fail—the adhesive forces are low. A further challenge with hydrophilic bioadhesives is their washout during in vivo application before crosslinking takes place. Moreover, hydrophilic adhesives have poor water resistance to avoid water swelling or diffusion, and the adhesion performance in wet condition or underwater dropped significantly after 24 h or longer.

One strategy to address this challenge is to design hydrophobic bioadhesives that can repel water from the interface and establish a strong wet/underwater adhesion. Although the contribution of hydrophobic components are recognized in mussels' wet adhesion, hydrophobic adhesives without hydrophilic components have not been explored for bioadhesives [24-26], since they have some intrinsic drawbacks, including: (i) water-immiscible hydrophobic fluid tends to form isolated droplets in water due to capillary break-up from interfacial tensions [27]; (ii) non-specific hydrophobic interactions are not as strong as covalent bonds [28]; (iii) diffusion of adhesive molecules is required to build strong interactions [29], while most highly mobile hydrophobic materials are small organic molecules with cytotoxicity; and (iv) rapidly curing is required.

Recently, some hydrophobic bioadhesives were reported for wet/underwater environments, while they still contain some hydrophilic components. Lang et al. proposed a hydrophobic liquid polyester prepolymer adhesive, which could be applied on wet biological tissue surfaces [30]. However, its adhesion strength to tissues was generally low (ca. 20 kPa) and primarily depended on non-covalent hydrogen bonding interactions. In addition, the underwater performance adhesion and water-resistant performance were not investigated. Han et al. reported a Fe³⁺ induced dynamic hydrophobic hydrogel with an underwater adhesion strength of ˜20 kPa, which is mainly attributed to the non-specific interactions from C18 long aliphatic chains with substrates [31]. In addition, the underwater adhesion performance of above hydrophobic adhesive decreased obviously with longer soaking time, which may be attributed to the presence of their hydrophilic components influencing the overall water-resistance. A hyperbranched liquid polymer-based underwater adhesive containing hydrophobic backbone and hydrophilic catechol side branches was also reported, which could be solidified in water through spontaneous coacervation driven by hydrophobic aggregation; however, the initial adhesion is low, and a longer curing time of 2 h is needed [9]. Collectively, literature reports to date indicate that in-situ adhesives with short curing time, strong instant underwater adhesion, and high water resistance are still elusive. Therefore, designing wet/underwater adhesives driven by the hydrophobic strategy is still challenging, especially for achieving strong instant adhesion under wet/underwater environments.

In order to form strong bioadhesion in wet/underwater conditions, we designed water-immiscible hydrophobic fluid adhesive with high mobility and low surface energy to repel surface water through hydrophobic exclusion. The underwater and in-situ applicable hydrophobic adhesive (UIHA) reported herein diffused on the irregular surface of biological substrates and form instantly strong interactions through mechanical interlocking when crosslinked. Furthermore, UIHA kept excellent water resistance attributed to hydrophobicity, and underwater adhesion was further enhanced via covalently bonding with substrates. The designed highly entangled hydrophobic macromolecular fluids in UIHA are comprised of three components: (i) macromolecular silicone fluid providing dynamic entanglement to prevent underwater capillary breakup, (ii) reactive polydimethylsiloxane (PDMS) precursor for gelation, and (iii) a small amount of silane to covalently bridge the hydrophobic-hydrophilic interfaces [32]. Silicone has excellent tissue and blood biocompatibility, low cytotoxicity, and is widely used as a medical filling/implant material, in drug delivery, and in wound care [33-38]. In spite of these features, silicone fluids have not been investigated for their potential us in underwater bioadhesives. Herein, we designed hydrophobic underwater adhesive based on silicone fluid because (1) highly flexible backbones for high mobility, (2) high hydrophobicity for good water resistance, and (3) low surface energy for repelling water from the substrate. The designed molecular composition of UIHA is different from regular silicone sealants, containing silane coupling agents and large amounts of entangled silicone macromolecules. Silane coupling agents were used as adhesion booster through pre-treatment of substrates' surface before applications of adhesives [39], but never for tissue substrates. They were rarely combined and used with PDMS simultaneously [38], which is not suitable for rapid curing. Although this impacts the curing efficiency and the adhesive's cohesion it fulfills the specific requirements for underwater in-situ applications, enhances underwater adhesion by building covalent bonding with substrates in one pot, and improves the overall underwater adhesion performance.

### SUMMARY OF THE INVENTION

According to a first aspect of the invention, there is provided a hydrophobic adhesive comprising:
a) about 25 wt % to about 75 wt % crosslinker;
b) about 25 wt % to about 75 wt % entanglement fluid; and
c) about 0.05 wt % to about 0.3 wt % hydrosilylation agent.

### BRIEF DESCRIPTION OF THE DRAWINGS

FIG. 1. Gelation behavior of macromolecular hydrophobic silicone fluids. (a) Critical concentrations of PDMS precursor in silicone fluids with different viscosity. (b) Time sweep rheological profiles of PDMS precursor-silicone fluid mixtures at their respective critical gelation concentrations (12 wt % for silicone 200, 20 wt % for silicone 10 k, and 25 wt % for silicone 500 k). (c) Time sweep rheological profiles of PDMS (Eco0035) precursor, silicone fluid/PDMS precursor mixture (25/75, wt %), and silicone 500 k/PDMS precursor mixture (25/75, wt %) containing 0.1 wt % VTMS. (d) Time sweep sweeping rheological profiles of PDMS precursors containing different concentrations of silane. (e) Time sweep rheological profiles of silicone fluid 500000/PDMS precursor mixture (25/75, wt %) containing 0.1 wt % VTMS in dry condition or underwater. (f) Gelation time vs. gelation temperature profiles of PDMS precursors and silicone 500 k/PDMS precursor mixture (25/75, wt %) containing 0.1 wt % VTMS, respectively. (g) The hydrolysis kinetic of VTMS silane in an immiscible binary phase system of hydrophobic CDCl₃/water. (h) Dynamic strain amplitude test of silicone 500 k/PDMS precursor (75/25, wt %) at the shearing rate of 10 rad/s with alternating strain amplitudes of 1% strain and 400% strain. (i) Dynamic strain amplitude test of silicone 500 k/PDMS precursor (65/35, wt %) at the shearing rate of 10 rad/s with alternating strain amplitudes of 1% strain and 400% strain. (j) Dynamic strain amplitude test of silicone 200/PDMS precursor (75/25, wt %) at the shearing rate of 10 rad/s with alternating strain amplitudes of 1% strain and 400% strain. (k) Photographs showing self-healing behavior of highly entangled macromolecule silicone gel (PDMS precursor/silicone 500 k=25/75, wt %). (11) and (12) The SEM images of PDMS-silicone gel (25/75, wt %) morphology after silicone fluid was extracted using hexane.

FIG. 2. Underwater in-situ application of UIHA. (a) The viscosity (as the shear rate approaches zero) vs. PDMS precursor weight fraction (inset shows the photograph of PDMS precursor and UIHA written respectively on glass substrate immersed in water). (b) Digital photograph showing underwater writing of UIHA. (c) The curves of viscosity vs. strain amplitude of various silicone fluid mixtures, including PDMS precursor, silicone 10 k, silicone 500 k/PDMS A (25/75, wt %. During the test, only Eco0035 (PDMS) A rather than Eco0035 A/B was used to avoid curing of samples. Here PDMS A and B has similar viscosity profiles), silicone 500 k/PDMS A (25/75, wt %), and silicone 500 k. (d) Scheme showing measurement of normal stress of silicone mixture droplets engaging/disengaging a stainless steel plate at a constant speed of 10 μm/s. The normal stress of liquid droplets during plate engagement and disengagement at dry condition or underwater, and liquid droplets includes (e) PDMS precursor (for the engagement/disengagement experiments, to prevent curing of liquid PDMS precursors during experiment, only Eco0035 (PDMS) A was used for experiment rather than the mixture of Eco0035 A and B, as Eco0035 A and B have similar viscosity profiles), (f) silicone 500 k/PDMS (25/75, wt %), (g) silicone 500 k/PDMS (25/75, wt %), and (h) silicone 500 k, (i) The normal adhesion stress of silicone mixture liquids on stainless steel plate underwater or in dry condition (**: p=0.0049; ***: p=0.0005; ****: p<0.0001, ns: p=0.9703 (silicone 500 k); ns: p=0.2254 (silicone 500 k: PDMS=50:50); ns: p=0.7925 (silicone 500 k: PDMS=25:75), n=3). (j) Under a water pressure of ˜5 psi, a polypropylene tube with punctured holes was in-situ sealed with UIHA. (k) Underwater directly sealing/isolating leakage of a working circuit.

FIG. 3. The dry or underwater adhesion performance of UIHA adhesives. (a) The time-lapse photographs showing the underwater in-situ adhesion of a cotton string with a weight of 50 g adhered to a glass substrate in 5 min. (b) The photographs showing that the strong underwater in-situ adhesion of the UIHA adhesive applied on glass substrate (25 mmx 16 mm) to successfully afford a weight of 5 kg. (c-e) The lapse shear adhesion of PDMS precursor/silicone 500 k mixtures with different weight fractions (25/75, 50/50, 75/25) on (c) glass substrate (*: p=0.0136; ***: p=0.0084, n=3), (d) PDMS substrate (****: p<0.0001, 20 n=3), and (e) porcine skin tissue substrate (*: p=0.0312; ***: p=0.0001, n=3) respectively. (f) The lap shear adhesion stress-strain curves of UIHA adhesives containing different amounts of silane on glass substrate. (g) The lap shear adhesion strength of UIHA adhesives containing different amounts of silane on glass substrate (*: p=0.0154; **: p=0.0024; ns: p=0.3722, n=3). (h) The lap shear adhesion stress-strain curves of UIHA, coated and stored in dry condition or underwater for different periods. (i) The lap shear adhesion of UIHA (0.1 wt % silane) and UIHA without silane (control group) soaked underwater for different periods on glass. The samples were soaked underwater up to 2 days (n=3 for each group. ns (UIHA without silane at different time): p=0.50957. ns (UIHA at different time): p=0.10363, ** (as prepared): p=0.00675, ** (2d): p=0.00248. ***: p=0.00038). (j) The lap shear adhesion stress-strain curves of UIHA without or with silane (0.1 wt %) on porcine skin tissue substrates stored underwater for a different period. (k) The lap shear adhesion of UIHA (0.1 wt % silane) and UIHA without silane (control group) soaked underwater for different periods on porcine skin substrate. The samples were immersed underwater up to 2 days (n=3 for each group, ns: p=0.30014; *: p=0.01389; ** (1d): p=0.00196; ** (UIHA between as prepared & ld): p=0.00196; *** (2d): p=0.00016; *** (UIHA between as prepared & 2d): p=0.00024). (1) The lap shear adhesion of cyanoacrylate, fibrin glue, and UIHA on porcine skin tissue stored underwater for 48 h (***: p=0.0003: ****: p<0.0001, n=3). (m) The Lap shear adhesion strength of current existing tissue adhesives on porcine skin substrates under dry conditions, including GelCORE [50], Evicel [50], CoSEAL [50], mussel inspired nanoparticles [51], TISSEAL [52], amino acid-based poly (ester urea)-catechol (polyester-DOPA) [52], Dermabond [1]. Histoacryl [1], Cross-linkable DOPA containing terpolymer adhesives (PAA-NHS-DOPA-thiol-PEG) [53]; or stored under wet conditions for various time, including poly (ester urea)-dihydroxyphenylalanine (PEU-DOPA) (wet, 4 h) [54], Fibrin glue (underwater 2d, measured), mussel based bioadhesives (underwater 2 h) [55], dry double side tape (DST, wet, 1 minute) [1], α-cyanoacrylate (underwater 2d, measured) and UIHA (this work, underwater 2d, measured). FIG. 4. In vitro and in vivo UIHA adhesion to arteries. (a) Burst pressure of UIHA, UIHA underwater for 24 h, fibrin glue, and fibrin glue underwater for 24 h (n=3). (b) SEM images of the interface between the porcine artery and UIHA (ex vivo). (c) Rat carotid artery incisions sealed by UIHA in vivo (n=8). Incisions before (i) and after sealing on day 0 (ii) and day 56 (iii). (iv) Micro-computed tomography angiography at day 3, showing the blood flow in the UIHA sealed site (left) and the normal control without operation (right). (v) Ultrasound image demonstrating UIHA-adhered carotid arterial wall and UIHA after 3 days. (vi) H&E staining of the adhesive interface between the rat artery and UIHA on day 56. (d) Fluorescent immunohistochemical analysis of the UIHA-sealed arteries after 56 days, showing no noticeable local macrophage (CD68) (i) and lymphocyte (CD3) infiltration (ii). Femoral artery incision sealed by UIHA on (e) rabbit and (f) beagle dog (n=3). Femoral artery incisions before (i) and after sealing (ii). (g) Porcine femoral artery incisions sealed by UIHA (n=5). Porcine artery incisions before (i) and after sealing on day 0 (ii) and day 56 (iii). Doppler images in cross-section (iv) and vertical section (v and vi) demonstrating vessel flow after sealing.

FIG. 5. Ex vivo and in vivo adhesive properties of UIHA on lung, skin and skull. (a) Ex vivo and in vivo tests on a rat lung leakage model sealed by UIHA-coated patches (n=5). (i) H&E and (ii) SEM images of the interface between lung and adhesives. Cut sites (iii) before and (iv) after sealing. On day 7, images of the lung leakage sites (v) with UIHA. (b) In vivo test on a porcine lung leakage model sealed by a UIHA-coated patch (n=3). UIHA patch (ii), cut sites (i) before and (iii) after sealing. (c) Ex vivo and in vivo tests on a rat skin incision sealed by UIHA (n=5). (i) H&E and (ii) SEM images of the adhesive interface on rat skin. Cut sites before (iii) and after sealing (iv). Images of the skin leakage sites (v) with UIHA and (vi) without UIHA sealing on day 5. (d) In vivo test on a skull injury sealed by UIHA (n=5). Skull injury (i) before and (ii) after sealing. Micro-CT images of bone integration and reconstructive (iii) with UIHA sealing after one month.

FIG. 6. Underwater adhesion mechanism illustration of UIHA adhesive. (a) The in-situ adhesion process of UIHA underwater, and (b) the potential applications of UIHA.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

As used herein, “about”, in reference to a numerical value, refers to a range of numerical values that are within plus or minus 10%, or plus or minus 5%, of the recited numerical value.

Building strong underwater bioadhesion is important for several applications but proved to be an extremely challenging task. In a wet/underwater environment, the water boundary layer hinders interfacial adhesion, which is further undermined by water-induced swelling in bioadhesives.

In order to form strong bioadhesion in wet/underwater conditions, we designed water-immiscible hydrophobic fluid adhesive with high mobility and low surface energy to repel surface water through hydrophobic exclusion. The underwater and in-situ applicable hydrophobic adhesive (UIHA) reported herein diffused on the irregular surface of biological substrates and form instantly strong interactions through mechanical interlocking when crosslinked. Furthermore, UIHA kept excellent water resistance attributed to hydrophobicity, and underwater adhesion was further enhanced via covalently bonding with substrates. The designed highly entangled hydrophobic macromolecular fluids in UIHA are comprised of three components: (i) macromolecular silicone fluid providing dynamic entanglement to prevent underwater capillary breakup, (ii) reactive polydimethylsiloxane (PDMS) precursor for gelation, and (iii) a small amount of silane to covalently bridge the hydrophobic-hydrophilic interfaces [32]. Silicone has excellent tissue and blood biocompatibility, low cytotoxicity, and is widely used as a medical filling/implant material, in drug delivery, and in wound care [33-38]. In spite of these features, silicone fluids have not been investigated for their potential use in underwater bioadhesives. Herein, we designed hydrophobic underwater adhesive based on silicone fluid because of (1) highly flexible backbones for high mobility, (2) high hydrophobicity for good water resistance, and (3) low surface energy for repelling water from the substrate. The designed molecular composition of UIHA is different from regular silicone sealants, containing silane coupling agents and large amounts of entangled silicone macromolecules. Silane coupling agents were used as adhesion booster through pre-treatment of substrates' surface before applications of adhesives [39], but never for tissue substrates. They were rarely combined and used with PDMS simultaneously [38], which is not suitable for rapid curing. Although this impacts the curing efficiency and the adhesive's cohesion it fulfills the specific requirements for underwater in-situ applications, enhances underwater adhesion by building covalent bondings with substrates in one pot, and improves the overall underwater adhesion performance.

In some embodiments of the invention, there is provided a hydrophobic adhesive comprising or consisting essentially of or consisting of:
a) about 25 wt % to about 75 wt % crosslinker;
b) about 25 wt % to about 75 wt % entanglement fluid; and
c) about 0.05 wt % to about 0.3 wt % hydrosilylation agent.

In some embodiments of the invention, the hydrophobic adhesive comprises or consists essentially of or consists of:
a) about 50 wt % to about 75 wt % crosslinker;
b) about 25 wt % to about 50 wt % entanglement fluid; and
c) about 0.05 wt % to about 0.15 wt % hydrosilylation agent.

In some embodiments of the invention, the hydrophobic adhesive comprises or consists of or consists essentially of:
a) about 75 wt % crosslinker;
b) about 25 wt % entanglement fluid; and
c) about 0.1 wt % hydrosilylation agent.

As discussed herein, the hydrophobic adhesive may be formulated as a spray, as a gel, for syringe application or as a patch.

Furthermore, as discussed herein, the hydrophobic adhesive may be used in “underwater” environments, that is, in a wide range of medical applications, as discussed herein.

The crosslinker may be PDMS or a modified PDMS.

The modified PDMS may be functionalized PDMS or curable PDMS.

The functionalized PDMS may contain at least one functional group selected from vinyl groups, methacrylate, acrylate, azide and alkyne.

The curable PDMS may be selected from platinum catalyzed PDMS, UV-curable PDMS, peroxide catalyzed PDMS, and PDMS crosslinked through click chemistry.

The entanglement fluid may be a silicone with a viscosity above 10000 cS, for example, a silicone with a viscosity of at least 500000 cS.

The entanglement fluid may be a silicone with a molecular weight greater than 60000 Da, for example a molecular weight between 60000 Da to 500,000 Da or higher.

The hydrosilylation agent may be a silane.

The silane may be vinyltrimethoxysilane, vinyltriethoxysilane, 3-(trimethoxysilyl) propyl methacrylate, allyltrimethoxysilane, or (3-mercaptopropyl) trimethoxysilane.

In some embodiments, the silane is vinyltrimethoxysilane or vinyltriethoxysilane.

Specifically, in contrast to current hydrophilic bioadhesives, we here describe an underwater and in-situ applicable hydrophobic adhesive (UIHA). We report polydimethylsiloxane (PDMS) tangled with macromolecular silicone fluid as a dissipation phase to provide strong interface adhesion strength. The silicone fluid repels the surface boundary water layer, overcomes capillary break up in water, and rapidly gelates with PDMS, while a small amount of silane (<0.2%) can bridge the hydrophobic adhesive and tissue substrates to an exceptional underwater adhesive strength. UIHA presents in-situ and instant adhesive performances when tested on artery, lung, bone, and skin tissues.

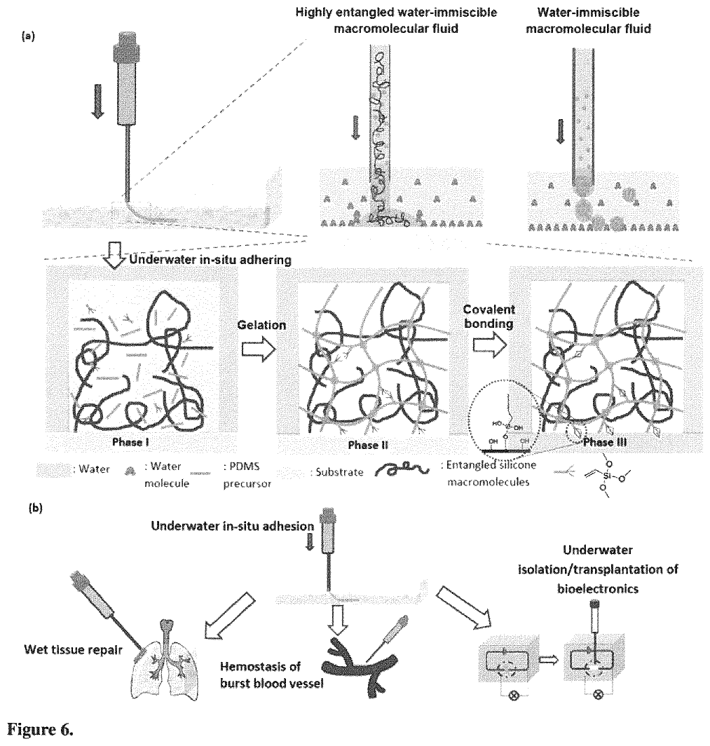

The adhesion process of UIHA involves three steps, as shown in FIG. 6(a). In step I, the entangled macromolecular hydrophobic fluid (PDMS precursor and silicone oil) is injected into water, touches the substrate, displaces water (the weak boundary layer), and spreads on the surface owing to the low surface energy of silicone [40]. For spreading on underwater substrates, the interfacial energy between solid/water (γ_{sw}) should be higher than the overall interfacial energy from infused liquid/solid (γₛₗ) and water/liquid (γ_{lw}), as shown in eq.1.

$$Spreading coefficient : S = {γ}_{s w} - {γ}_{sl} - {γ}_{lw} \geq 0 \qquad ( eq . 1 )$$

Despite low surface energy, partial wetting of silicone fluid was found on solid surface underwater [41]. The fluid is confined in the applied areas, as the spreading is suppressed significantly by the viscous dissipation of the highly viscous liquid. According to molecular-kinetic theory [42, 43], the contact line friction (§) is proportional to liquid viscosity and exponentially related with reversible work of adhesion (γ(1+cosθ⁰)), as shown in eq. 2 [44].

$$ξ \approx \frac{ην}{{λ}^{3}} exp ( \frac{γ ( 1 + cos {θ}^{0} )}{{nk}_{B} T} ) \qquad ( eq . 2 )$$

where η is liquid viscosity; v is liquid molecular flow volume; λ is the jumping length, n is the density of absorption sites, and kg is the Boltzmann constant and T is the temperature in Kelvin.

Following anchoring of the hydrophobic liquid thread, entangled bulky fluid continuously adheres to the substrate due to hydrophobic and viscous interaction. The capillary thinning behavior of a Newtonian viscous fluid is weakened by increased viscosity [45, 46]. Capillary thinning is more gradual in viscoelastic fluids due to retardation of high viscosity and stress from extensional deformation, which effectively hinders the capillary breakup of the hydrophobic fluid after extrusion. In step II, well-diffused fluid on the substrate solidifies through crosslinking of PDMS network, and the adhesion was mainly attributed to hydrophobic interaction and interfacial interlocking of adhesives with irregular substrate surface [47]. Then the interfacial adhesion is further enhanced by covalently bonding from hydrolysis of silane in step III.

A PDMS precursor, platinum catalyzed Ecoflex 0035 (a short curing time of 5 min), was employed to construct organogel network and silicone oil as a flow and tangled phase to consume dissipative energy. A small amount of vinyl trimethyl silane (VTMS: <0.2 wt %) was added, as vinyl groups could react with PDMS precursors through hydrosilylation reaction [32]. On the other hand, the silane groups could be hydrolyzed with water or hydroxyl groups on the substrate.

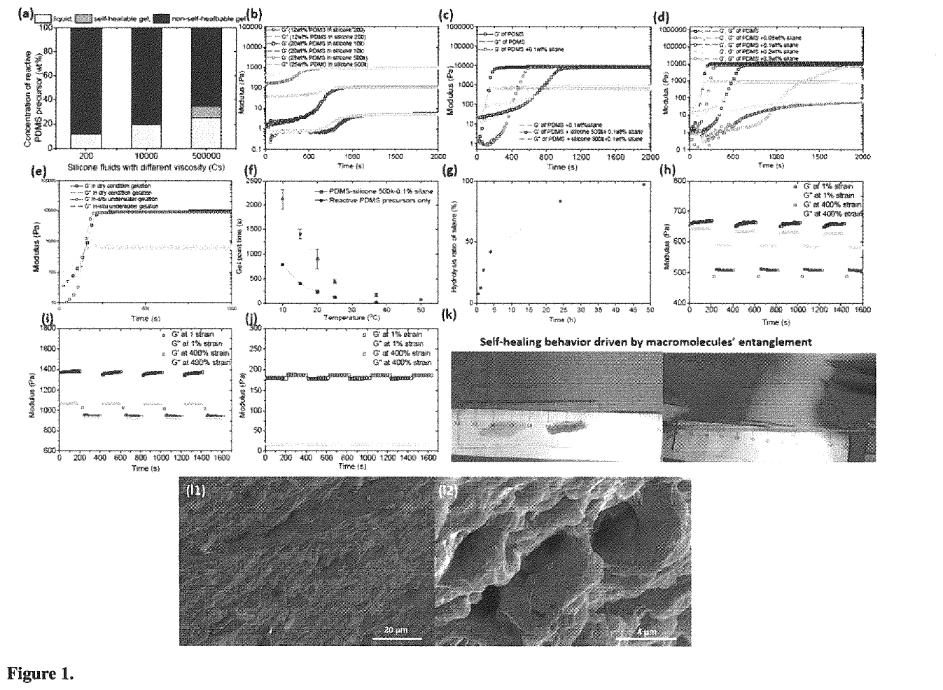

Unlike small molecules, gelation of macromolecular fluids shows obvious chain length dependence of silicon oil. Three types of silicone fluids with different viscosity (molecular weights) were chosen, including silicone 500,000 cs (silicone 500 k), silicone 10,000 cs (silicone 10 k), and silicone 200 cs (silicone 200). Silicone 500 k has a weight average molecular weight of ˜260 kDa, much higher than its critical entanglement molecular weight (29 kDa, M_{c.silicone}) [48], while molecular weights are ˜9.5 kDa and ˜60 kDa for silicone 200 and silicone 10,000 respectively [49]. As shown in FIG. 1(a)˜(b), the gelation was assessed by the crossover of the storage modulus (G′) and loss modulus (G″), which shows obvious molecular weight and concentration dependence. Among three silicone fluids, the lowest critical gelation concentration was 12 wt % for silicone 200, but 25 wt % for silicone 500 k, suggesting long-chain architecture hindered the formation of a crosslinked network. For reactive PDMS precursor, the gelation time was only ˜2 min at room temperature, which was delayed to ˜7 min with the addition of 0.1 wt % silane (to PDMS), as the hydrosilylation reaction of silane competes with crosslinking and slows the formation of PDMS network (FIG. 1(c)). With the addition of silicone 500 k, both G′ and G″ increased, but the gel point time was further delayed due to the steric effect. As will be known by those of skill in the art, the gel point time is important for application of the hydrophobic adhesive of the invention for example underwater as for example an in-situ adhesive. Specifically, the shorter this time period is, the faster the gelation/crosslink of the adhesive is, which is good for in-situ applications of the underwater adhesive, as discussed herein. Furthermore, while the introduction of entangled macromolecular silicone will postpone gelation, the gelation time is still within an acceptable range. The amount of silane also had a significant influence on UIHA gelation, which retarded gelation time and decreased gel modulus, as shown in FIG. 1(d), as some crosslink points in PDMS network were substituted by conjugation with silane. When the concentration of silane reached 0.5 wt % of PDMS precursor, the gel modulus dropped considerably. This is because, while introduction of silane will improve adhesion capacity, it will hinder the crosslinking of UIHA gel through its competing reactions with the crosslinking agent. Consequently, while an increase in silane content will enhance interfacial adhesion between gel and substrate, it will also reduce cohesion inside the gel. Thus, the adhesion capacity depends on both interfacial adhesion and inside cohesion of the adhesive gel. Therefore, the concentration of silane is an important parameter in UIHA adhesive, as discussed herein. The further the crosslinking between silane groups inside adhesives will take longer time due to lack of water, but may take place due to high mobility of PDMS chains under ambient temperatures. The storage modulus (G′) of UIHA gels increased by ˜27% when soaked underwater up to 2 days, suggesting the elastic crosslinking network became more well-developed over time, which may be due to the continuous crosslinking of silane groups inside UIHA.

The underwater gelation behavior of UIHA adhesives is critical for bioadhesive applications. FIG. 1(e) shows the gelation time, G′, and G″ are similar in a dry condition or underwater, indicating excellent water tolerance of UIHA. As the hydrosilylation reaction rate is temperature dependent, the gelation of UIHA became faster when the temperature increased. As shown in FIG. 1(f), the gelation of UIHA was 40 min at 10° C., 7 min at 25° C., 2 min at 37° C. and 0.4 min at 50° C. Therefore, two parts of UIHA were mixed and stored at low temperature for an extended period but were cured rapidly after application under body temperature (37° C.). With an external thermal source, the period of curing could be shortened to less than one minute. The temperature dependence and water insensitivity indicates the great potential of UIHA for emergency bleeding and injured tissue treatment. The hydrolysis kinetic of silane in a water/water-immiscible liquid binary phase system (water/chloroform-d) was examined 1H NMR (FIG. 1(g)).

In a highly entangled silicone fluid-PDMS gel, the PDMS covalent crosslink formed the primary elastic network, which was strong and non-recoverable. Specifically, as will be apparent to one of skill in the art, the crosslink network is essentially made of covalent bonds, which are non-reversible, which is important and could gelate the whole adhesive and provide strong adhesion (the covalent bond is chemical bonding, which is much stronger than physical entanglements). The entanglement of free silicone macromolecules formed a secondary dynamic network, which was weak but recoverable and self-healable, and therefore entangled silicone gels exhibited interesting self-healing behavior, as shown in FIG. 1(k). Two pieces of silicone 500 k-PDMS gels were easily merged together by gentle contact and the self-healing performance was determined by the fraction of the dynamic components. When the weight fraction of PDMS precursor was between 25˜35 wt %, entangled silicone gels showed typical self-healing behavior, in which G″ was higher than G′ under high strain amplitude region (FIGS. 1(h) and (i)). This is a unique property of UIHA adhesive with specific composition ratio of crosslink phase and free phase. This kind of self-healable adhesion property may be important in some applications, such as sealing or isolation of implantable bioelectronics in body or underwater in-situ isolation. The self-healing performance could perform better to prevent the leaking or short circuit of implants. With increasing PDMS weight fraction to 40 wt %, the entangled silicone gel became non-recoverable, as increased crosslink density of the primary non-recoverable network restrains mobility of entangled silicone fluid. That is, the crosslinking network restrains the mobility of free silicone fluid in the gel, as the system is gelled. The hydrophobicity of the fluid also prevents its spreading or diffusion in water. On the contrary, the gel with silicone 200 did not show the self-healing activity owing to less likely entanglement with shorter chains. The gelation of macromolecular silicone fluid also works for other PDMS precursors with various gelation time and modulus. The volume of the UIHA gel shrank ˜30% after silicone fluid was extracted by sonication in hexane, but still kept the typical porous morphology of bulky gels, as shown in the SEM images in FIG. 1(l).

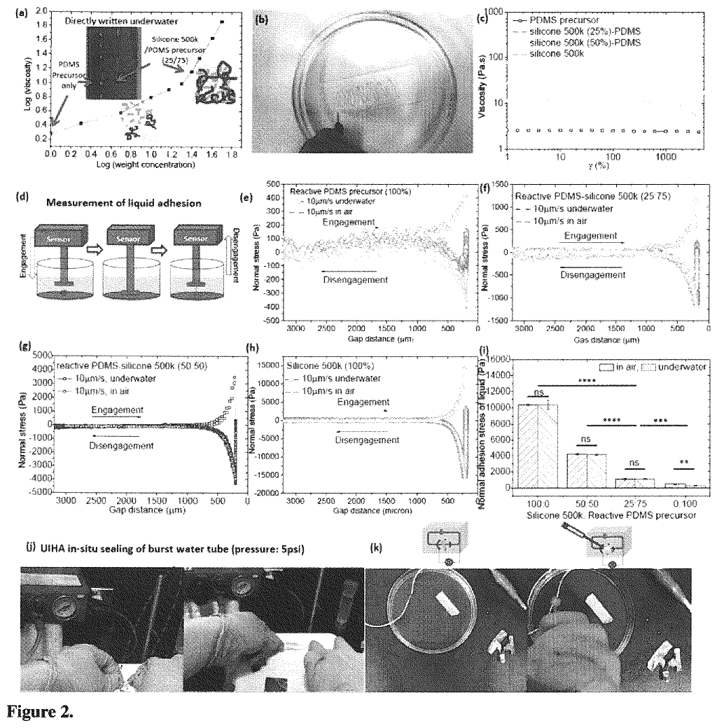

The highly entangled UIHA is suitable for underwater in-situ bioadhesion and surgical sealing and even electrical isolation. The water-immiscible entangled macromolecular fluid could form the continuous phase underwater with little influence of interfacial tension and corresponding capillary breakup owing to the high viscosity from the entanglement of macromolecular fluid. The critical entanglement concentration or weight fraction of high molecular weight silicone fluid in reactive PDMS precursors was determined by rheological tests, as shown in FIG. 2(a). As can be seen, both molecular weight and concentration Are important as with higher molecular weight, less concentration is needed. The range of concentration of high molecular weight silicone fluid is 25 wt %-75 wt %. The viscosity of silicone 500 k/PDMS mixtures under varying shear conditions showed shear-thinning viscoelastic behavior, as shown in FIG. 2(c). As viscoelastic fluids, when UIHA adhesive was ejected out to touch the substrate, the momentum would not be dissipated instantly, and the fluid would apply a certain pressure on the substrates to repel surface water layer and achieve close contact with the substrate surface. Specifically, when UIHA contacts a substrate, it will repel water away, as discussed herein. The underwater engagement pressure and the separation of normal stress between the moving plate and adhesive liquid was measured using an in-house built instrument, as shown in FIG. 2(d). FIG. 2(e)-(i) shows the normal stress of liquid adhesive and control groups on stainless steel substrate, which was engaged/disengaged by another steel plate at a constant speed of 10 μm/s. PDMS precursor showed very low pressure and break up adhesion on steel substrate due to its instant energy dissipation. With the addition of silicone 500 k, the highly entangled liquid shows typical viscoelastic behavior with the normal stress of 1000 Pa (25 wt % silicone 500 k) and 3000 Pa (50 wt % silicone 500 k), respectively. The adhesion of adhesive liquid (pre-gel) during disengagement in water or dry condition are similar, indicating the full contact of adhesive and substrate without boundary water barrier and completely water-repelling. As will be apparent to those of skill in the art, for underwater/wet adhesion, it is quite important to remove the surface water layer on a substrate, which if not removed, will hinder direct contact between adhesive and substrate and therefore weaken the interfacial adhesion significantly. However, in this case, the hydrophobic silicone fluid can repel water due to its low surface energy and water-immiscible hydrophobicity. The engagement/disengagement experiment further proves that the interaction between adhesive liquid and substrate are similar no matter if UIHA is coated in air or in water, indicating that the adhesive repelled water and fully contacted the substrate surface. Due to the excellent in-situ underwater adhesion, UIHA adhesive could be used. for example, for underwater in-situ electrical isolation and water burst sealing, as shown in FIGS. 2(j) and 2(k), which demonstrates applications for bioelectronics' implantation/sealing.

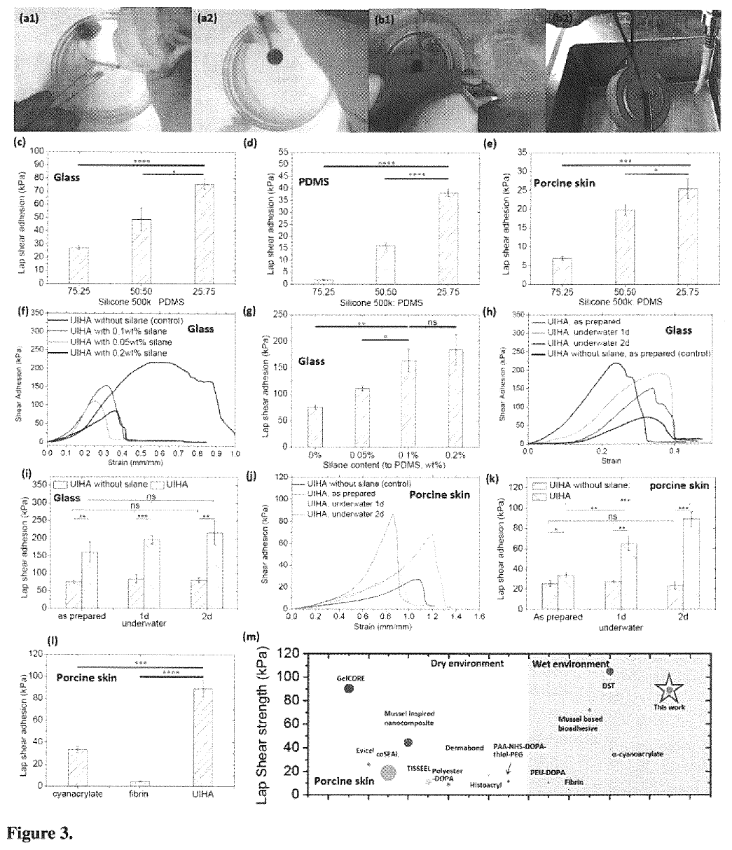

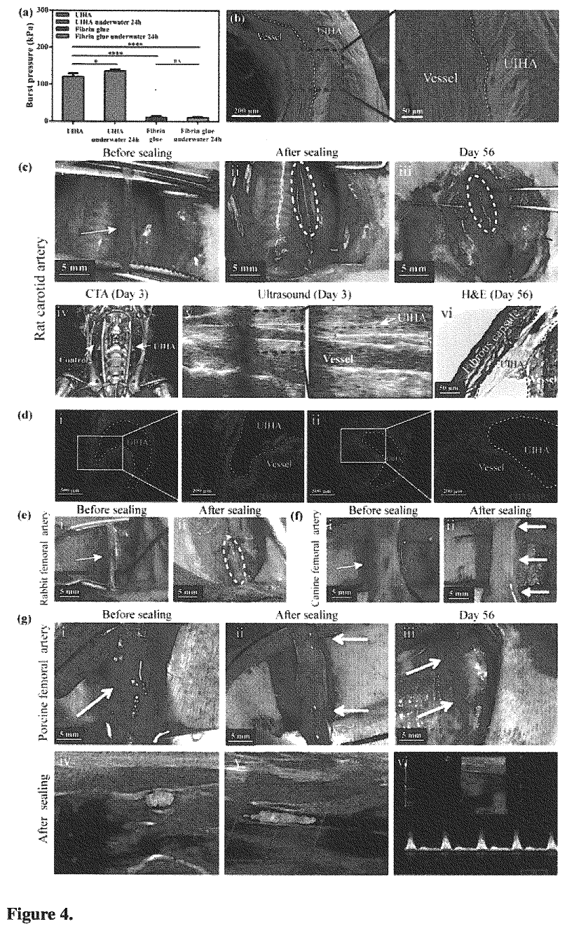

The UIHA shows impressive instant underwater adhesion performance in FIG. 3a, and the shear adhesion on glass (25 mm×15 mm) of underwater in-situ coated UIHA can afford a weight of 5 kg and water flow blast, as shown in FIG. 3b. The shear adhesion of UIHA without silane was assessed on glass, PDMS, and porcine skin substrates, respectively, suggesting PDMS-silicone 500 k (75:25, weight ratio) has an optimal shear adhesion (FIG. 3(c)˜3(e)). With the introduction of 0.1 wt % silane (to PDMS), the lap shear adhesion on glass doubled to 164.8+20.4 kPa in 15 min due to the covalent bonds between silane and abundant hydroxyl groups on glass, and the average values continued to increase but were not significant with extended time as shown in FIGS. 3(f) and 3(g). FIGS. 3(h) and 3(i) present excellent water resistance of UIHA, and water had little influence on the adhesive property of UIHA on the glass. As time went on, the shear adhesion strength of UIHA on glass soaked in water increased to 216±33.8 kPa, exhibiting excellent wet adhesion and stability. Unlike glass substrate, the instant adhesion of adhesive with silane on porcine skin was increased from 25.4±2.7 kPa to 34.0±2.3 kPa (˜33% improvement) with silane coupling agents (FIG. 3j& 3k). With long soaking time of 48 h, the underwater adhesion was tripled to 89.1±7.4 kPa, attributed to the covalent conjugation of silane and hydroxyl groups on porcine skin. To date, most bioadhesives (tissue adhesives) show poor water resistance, especially their wet/underwater adhesion dropped significantly after 24 h, which is a huge challenge for the applications of tissue adhesive. In contrast, our UIHA adhesive showed excellent water resistance and underwater adhesion due to its hydrophobicity and covalent bonding of reactive silane. For example, the UIHA soaked in water for 48 h reached a shear adhesion of 89 ka on porcine skin surface. The increase of adhesion strength on porcine skin substrate is slower than that on glass substrate, which may be due to slower formation of covalently bridging bonds on porcine skin, as glass substrate has more abundant hydroxyl groups (FIGS. 3(j) and 3(k)). Therefore, longer time is needed for silane on the interface to react with the porcine skin substrate to build stronger adhesion. The adhesion strengthens of UIHA with silane increased to ˜ 3 fold on both glass and porcine skin substrate after 2d, compared with the adhesive without silane. Compared with current bioadhesives, UIHA showed impressive shear adhesion strength in a wet environment and water stability on porcine skin substrates (FIGS. 3(l) and 3(m)).

Repair of arterial rupture and lung leakage is still a challenge for bioadhesives [1, 9, 14-23]. To evaluate the adhesive strength of UIHA, we first performed in vitro burst pressure test in blood. To evaluate the adhesive strength of UIHA, we first performed in vitro burst pressure test in a blood vessel and lung models. A punctured hole in 2 mm diameter on porcine skin was then placed on a pressure chamber and sealed by UIHA or fibrin glue. The sealed skin samples were then either kept underwater for 24 h or were kept in ambient conditions before burst pressure was measured. The burst peak pressures were shown in FIG. 4a. The burst pressure of UIHA was 120.60±9.36 kPa; its underwater burst pressure increased to 136.80±4.08 kPa after 24 h (p=0.0285). In contrast to fibrin glue with 11.27±3.79 kPa (p<0.0001) and 10.57±1.59 kPa (p<0.0001) underwater for 24 h, the UIHA showed superior sealing strength with around 12-fold enhancement. A very tight bonding occurred in the interaction interfaces between UIHA and porcine carotid artery ex vivo as shown by scanning electron microscopy (SEM) (FIG. 4b). Meanwhile, UIHA displayed superior anti-swelling ability in aqueous phase. For in vivo tests, UIHA was applied to the incision of the carotid artery in the rat model. The puncture created by a 25G needle (around 0.5 mm in diameter) in the vessel (around 1 mm in diameter) led to arterial blood spurted out. 10 μl UIHA was then applied to the puncture for 3 min (FIG. 4c, i and ii). After the vessel clamps were released, no visible leakage was detected. No artery hematoma was observed post-surgery 24 h. After 3 days, micro-computed tomography angiography (CTA-based) 3D reconstruction of the carotid artery demonstrated 100% patency for all UIHA-sealed arteries, similar to the normal artery (FIG. 4c, iv). On day 3, ultrasound (FIG. 4c, v) revealed that UIHA (around 0.386 mm in the thickest part) adhered to the wall of the vessel and no vascular stenosis or thrombus was formed. From H&E staining (FIG. 4c, vi), a thin fibrous capsule (with a thickness of about 50 μm) was observed around the UIHA-sealed vessels after 8 weeks. Immunohistological analysis did not show either CD68+macrophages (FIG. 4d, i) or CD3+ lymphocytes (FIG. 4d, ii) on day 56, suggesting no evident inflammatory reaction after 8 weeks.

Encouraged by the above results, we made a longitudinal incision by a scalpel in the femoral artery on rabbit (around 2-mm) (FIG. 4e, i and ii) and beagle dog (about 2-3 mm) (FIG. 4f, i and ii). As can be observed, UIHA blocked blood leakage efficiently. To further evaluate UIHA's strong adhesion, UIHA was applied to seal the porcine femoral artery incisions (FIG. 4g, i and ii). A 2-3 mm longitudinal incision was created by a scalpel, and blood was quickly spouted out from the defect. A UIHA solution was applied to seal the arterial injury (about 5-10 minutes). Hemorrhage was not observed after removing the hemostatic clips and hematoma was not detected within 24 h after surgery. Doppler imaging revealed the blood flow in the cross-section (FIG. 4g, iv) and vertical section (FIG. 4g, v), and thrombus formation was not detected on day 3. The average blood flow velocity of UIHA-sealed arteries (62.72±6.99 cm/s) was similar to that of sham group (69.82±3.78 cm/s, p=0.5606). Thus, the UIHA was able to effectively seal the artery by maintaining mechanical strength and biocompatibility.

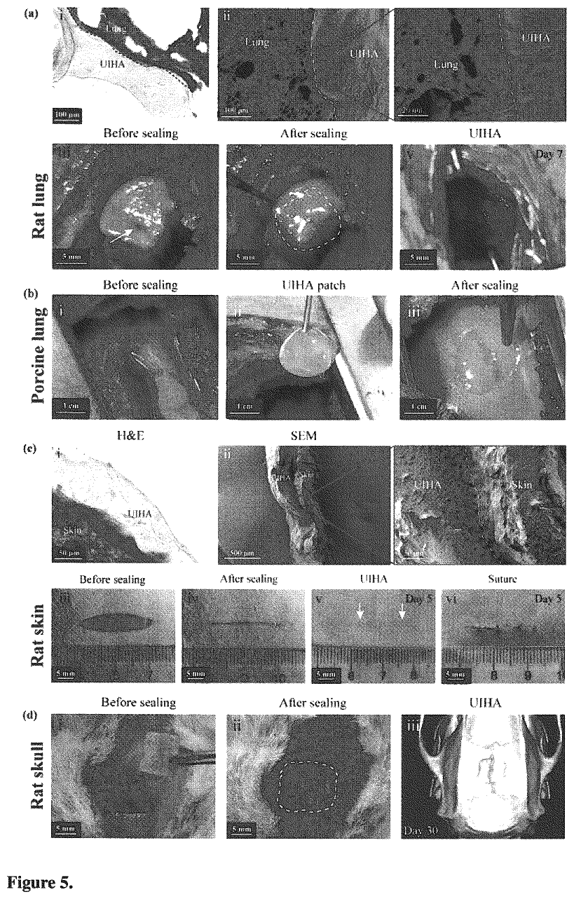

UIHA adhesion performance was also verified in lung, skin and skull bone. The tightly bonded interfaces were found between UIHA and lung (FIG. 5a, i and ii), as well as UIHA and skin (FIG. 5b, i and ii). UIHA can be built in a patch to be adapted to the complex physiological environment such as in lung where high pressure and hemorrhage, once a physical incision occurred, is fatal. Our patch (around 5 mm in diameter) combined with UIHA solution can seal the leaked lung effectively and stop the bleeding in the presence of burst pressure (FIG. 5a, iv). After 7 days, UIHA remained adherent to the wound site (FIG. 5a, v) and the in-situ water immersion test showed absence of air bubbles. Based on UIHA-sealed lung leakage on rats, we made an incision on the porcine lung by a scalpel (around 1-cm) (FIG. 5b, i). UIHA patch (approximately 12 mm in diameter) and UIHA liquid were combined (FIG. 5b, ii and iii) to seal the leaking porcine lung where air leakage and bleeding were stopped effectively. In order to demonstrate the potential application on skin, a cut of 2.5 cm transverse incision on rat abdominal skin was made, where a large tension environment could be built (FIG. 5b, iii). UIHA can completely close the wound gap (FIG. 5b, iv). Wounds with UIHA (FIG. 5b, v) presented satisfying healing compared with suturing groups (FIG. 5b, vi). UIHA can also seal hard tissues, such as rat skull (FIG. 5c, i and ii). Micro-CT revealed that the skull crack diminished during bone regeneration and the growing integration with the host over 30 days (FIG. 5d, iii). Moreover, UIHA was found to be non-cytotoxic and evoked minimal host inflammatory response at the interface of UIHA and tissue.

In conclusion, the unique hydrophobic UIHA exhibited exceptional adhesion for in-situ hemostasis and tissue repair for artery, lung, bone, and skin. In UIHA, non-hydrophilic elastomer interwoven with macromolecular organic viscous fluid created an in-situ underwater tissue/organ sealing and wound closure capability [7, 56]. The underlying mechanism sheds light on the design and strategic development of tissue sealants, surgical glue, and even implantation of bioelectronics under extreme environments.

### Materials and Methods

Ecoflex-0035 (E35A/E35B, AB components curable PDMS, platinum catalyzed, Smooth-on Company), Ecoflex-0050 (E50A/E50B, AB components curable PDMS, platinum catalyzed, Smooth-on Company), Slygard 184 (PDMS, Dow Corning), silicone fluid (viscosity: 500000 mm²/s, Beijing Haibeisi Tech, Silicone-500 k), silicone fluid (viscosity: 10000 mm²/s, Beijing Haibeisi Tech, Silicone-10 k), silicone fluids (viscosity: 200 mm²/s, Beijing Haibeisi Tech, Silicone-200), vinyltrimethyl silane (VTMS, Sigma Aldrich), fresh porcine skin tissue (purchased from local meat supermarket, stored in a −20° C. freezer before use), α-cyanoacrylate (Guangzhou Baiyun Medical Glue Company), fibrin glue (Guangzhou Beixiu Biotechnology).

### Preparation of Underwater and In-Situ Hydrophobic Adhesive (UIHA)

All the procedures were performed in an ice bath, and all materials were pre-cooled on ice. In a typical preparation, 50 mg of VTMS and 950 mg of Ecoflex-0035 B (E35B) were mixed in a 2 ml polypropylene centrifuge microtube to obtain a mixture containing 5 wt % silane, and the microtube cap was tightly sealed until used. 750 mg of E35A, 500 mg of silicone 500 k, and 30 mg of the above mixture containing 5 wt % silane and 720 mg of E35B were weighed into another 2 ml polypropylene microtube mixed in sequence. The mixture was well-mixed with a thin rod in an ice bath, and then centrifuged for 15 sec at 5000 rpm to remove the bubbles to obtain the UIHA containing 0.1 wt % silane (0.1 wt % is the weight ratio of silane to the sum of E35A and E35B). Freshly prepared adhesive mixture was s used immediately for all experiments.

### Rheology Characterization

All rheological measurements were conducted on a TA rheometer (Discovery Hybrid HR-1) equipped with a stainless steel cone plate) (2° angle of 20 mm diameter geometry or a flat plate of 8 mm diameter geometry. For the time-sweeping tests, samples endured a constant shearing rate of 10 rad/s with a strain of 0.5% under various temperatures from 10° C. to 50° C. To measure the underwater gelation behavior, the plate was immersed in the water reservoir of ˜5 mm depth, and the measurements were performed when water temperature reached equilibrium with the rhemeter set temperature.

In the oscillation frequency sweeping tests, the frequency swept from 0.001 rad/s to 1000 rad/s at 25° C. In the viscosity measurements of the mixtures of reactive PDMS precursor/silicone fluids, to prevent increasing of viscosity due to crosslinking reactions during measurements, only E35A was mixed with silicone fluids rather than E35A/B components, as E35A and E35B have similar viscosity and rheological profiles.

In the strain alternating experiments, all gel samples were performed under a constant shear rate of 10 rad/s with the strain alternating between 0.1% and 400%. The period of every step is 200 sec, and there were 8 steps/4 cycles in total.

The strain sweeping experiments were also performed under similar conditions, the strain ramped from 1% to 4000% under a constant shear rate of 10 rad/s.

The underwater engagement/disengagement experiments were implemented on rheometer either, the bottom plate is flat, and upper plate is the steel stainless cone plate (2º) angle of 20 mm diameter geometry (the plate was considered as flat plate for calculation). The initial gap between two plates is 3.2 mm, and 150 μl of PDMS/silicone liquid mixture was added onto the center of bottom flat plate to ensure the liquid could fill the whole gap when gap distance is minimum. During experiments, the upper plate was approaching the bottom at a speed of 10 μm/s until the minimum gap reached 200 μm, and then the upper plate started to disengage.

The storage modulus (G′) of UIHA gel soaked underwater over time were measured through oscillation time sweep rheological tests. The UIHA gels were cured at 37° C. for 30 mins for complete crosslinking of ecoflex 0035A and 0035B before test. All samples (20 mm diameter and 300 μm gap distance) were tested at 25° C. with a constant strain of 0.5% and a shear rate of 10 rad/s. Then all samples were soaked underwater and measured again under same conditions after 1d and 2d respectively. The water on samples' surface were wiped off by paper towel and samples were further dried under vacuum for 1 h before experiments to remove surface water completely.

### ¹H NMR Characterization

All ¹H NMR characterizations were carried out on an Avance300 spectrometer. Samples were dissolved in deuterated chloroform at a concentration of ˜ 10 mg/ml. To evaluate the hydrolysis behavior of VTMS in a hydrophobic/hydrophilic immiscible binary phase system, 100 mg of VTMS was dissolved in 2 ml of CDCl₃ in a 50 ml centrifuge tube, and then 40 ml of DI water was poured into the tube. The mixture was then left standing for layer separation. At different time points, the aliquots were collected from CDCl₃ layer and diluted to 10 mg/ml, and dried with anhydrous sodium sulfate powder before NMR characterization.

### FTIR-ATR Characterization

FTIR characterizations were performed on a Thermo Scientific Nicolet Is10 FTIR spectrometer equipped with an ATR accessory. The resolution is 4 cm⁻¹ and the number of scans is 4. The porcine skin tissue sample was cut to thin strips, and fats were removed by razor blades as much as possible. After repeatedly washing with water, the porcine skin strip was lyophilized to remove water completely.

### Scanning Electron Microscopy (SEM) for Gel Morphology Characterization

The SEM characterization of gel morphology was conducted on a FEI Nova NanoSEM 450 with an operation voltage of 15V. To remove the silicone fluid in the tested gels, samples were washed by sonicating in hexane in a bath sonicator for 3 h per day for 5 days with solvent exchanging twice a day. A notch was cut on sample edge and then the sample was teared apart. Samples were sputter-coated by a thin layer of gold for SEM imaging.

### Lap Shear Adhesion Test

All the lap shear adhesion tests were performed using an Instron Universal tester (Instron 5965) equipped with a loading cell of 1 kN. For all sample substrates, the surface was cleaned with ethanol and DI water before coating. For the test on glass substrates, thin cover glass slides (22 mm×22 mm,) were used as substrates, and ˜25 μl sample was coated on the area of 22 mm×4˜6 mm and cured for 15 min at 25° C. The ends of glass slides held by clamps were taped with paper to prevent slipping during test, and two clamps should be aligned to avoid internal stress. For tests on PDMS substrates, Sylgard 184 was used to prepare the PDMS substrate in accordance with the product instruction. The Sylgard 184 PDMS were cut to strips of 50 mm×10 mm, and ˜25 μl mixture was applied onto a region of 10 mm×10 mm for each sample. For tests on porcine skin tissue, porcine skin was thawed first, and then cut to strips of 50 mm×10 mm. The fat tissue and hair on porcine skin substrates were removed with razor blade as much as possible. The cleaned porcine skin tissue strips were soaked in DI water and stored in a fridge at 4° C. before use. To prepare samples, ˜100 μl mixture was coated on the area of 10 mm×10 mm, and an external pressure of ˜600 Pa was applied on each sample to prevent the porcine skin strip bending. Samples were fully cured at 25° C. for 15 min and then tested directly or soaked in water for certain periods before test. For each sample group, n=3.

### Underwater In-Situ Sealing/Isolation of Electronics

The underwater in-situ isolation model of electronics was built in-house. A circuit (3V) with broken isolation layer was soaked in salt water (1M CaCl₂) solution), which bridges the leaking area and another LED bulb indicator to form another circuit. To isolate the leaking area underwater, prepared UIHA adhesive was injected onto the broken isolation layer area to re-seal the electronic circuit completely.

### In-Situ Seal of Water Burst of Balloon and Tube

A water balloon was prepared by filling nitrile latex with water. One hole was created on the balloon by puncture with a needle (20G). An UIHA patch (15 mm×15 mm) was prepared in advance, and then coated with a thin layer of UIHA liquid. The UIHA patch was adhered to the hole and gently pressed by finger for a few seconds to stop water leaking.

The burst model was built with an air compressor, an air pressure controller connected with a polypropylene (PP) tube (Inner diameter: 3 mm; outer diameter: 4 mm). A ˜5 psi pressure was applied to a red color water-filled PP tube and the other end of tube was sealed completely. One punctured hole was created on one side of the PP tube by a needle of 20G which led to water shooting out. The system was set on a hotplate of 37° C. to mimic body temperature environment. An UIHA patch (˜10 mm×˜5 mm) was prepared in advance, and then coated with a thin layer of UIHA liquid. The patch was adhered to the hole area of the PP tube, and gently pressed manually for 2 min to seal the broken tube.

### Ex Vivo Burst Pressure Measurements

Ex vivo burst pressure of UIHA was obtained by following standard protocol for measuring surgical sealants. It was performed on a custom-made pressure chamber equipped with a digital manometer and a syringe pump. Porcine skin tissues were purchased from a local market. The adipose tissue was removed and a 2 mm diameter punctured hole was created. 200 μL of adhesive solution was injected onto the defect through a syringe. Samples were fully cured at 37° C. for 30 min and then tested directly or soaked in water for certain periods before testing. After gelation, the pressure was applied by pumping PBS via a syringe pump at a rate of 0.75 ml/min, and the pressure was recorded by the manometer.

### SEM Characterization of Tissue-Adhesive Interface

Samples with the surrounding tissue were fixed overnight with glutaraldehyde and lyophilized. The samples were then mounted onto an aluminum holder and sputter-coated with gold. SEM images of the samples were obtained on an emission scanning electron microscope (ZEISS crossbeam 340-47-76) at 10-20 kV.

### Animal Experiments

All animal experiments were carried out in accordance with the regulations of ethical approval for research involved animals and were approved by the Ethics Committee of the Third Military Medical University, China.

### In Vivo Biocompatibility and Biodegradation of UIHA

Subcutaneous implantation was carried out with male Sprague Dawley rats (200-250 g). Rats were anesthetized with 1-1.5% isoflurane. 1.5 cm incisions were made and separate subcutaneous pockets were created on the dorsum of the rat. The implanted materials were gelled, weighed, sterilized and implanted into the dorsal subcutaneous pockets (n=5, 30-60 mg). The skin incisions were closed by suturing. On day 14, 28, 56 and 84, the animals were euthanized by isoflurane (2.0 to 2.5%) inhalation, and the implants with the surrounding tissue were explanted for further histological analysis. For in vivo biodegradation evaluation, the surrounding tissue beside the implants was peeled off and then the residues were weighed. The degradation rate was measured based on the changes of weights before and after implantation, which was calculated with the following equation: (W_{before}−W_{after})/W_{before} in percentage.

Incision Closure of Rat Carotid Artery with UIHA

The incision sealing capacity of UIHA for carotid artery was tested on rats. Rats (n=8) were anesthetized as described previously. Under sterile conditions, the neck was incised, and the carotid artery was exposed and blocked by 2 vascular clamps. An incision was made in the vessel by a 25G needle. UIHA solution (10 μL) was applied to the incision. After UIHA gelation for 3 min, the vascular clamps were released, and the artery incision was closed by UIHA. After 3 days, micro-computed tomography angiography (CTA) and ultrasound with color Doppler (Visual Sonics, Vevo 2100) were performed to evaluate blood flow.

### Incisions Closure of Femoral Artery in Large Animals

Closure of femoral artery incisions were tested on rabbits, canines, and pigs. Anesthesia of rabbits (n=3), beagle canines (n=3), and mini pigs (n=5) were induced with pentasorbital sodium (1%) and then maintained by the inhalation of 2.0-2.5% isoflurane. Surgical preparations were performed as described previously. Briefly, the skin was incised, and femoral artery was exposed and controlled by two vascular clamps proximally and distally. A 2-3 mm incision was created with a blade scalpel. The UIHA solution (20 μl on rabbits, 200-300 μl on canines and pigs) was applied on the wound. After 5-10 minutes for UIHA curing, the vascular clamps were released and no bleeding was detected. 8 weeks later, ultrasound with color Doppler (Esaote Mylab system, Esaote) was performed to evaluate blood flow.

Leakage Sealing of Rat and Pocine Lung with UIHA

Lung leakage sealing capacity of UIHA was tested on rats (n=5) and mini pig (n=3). Anesthesia was performed as described above. Breathing was maintained by a ventilator. After a right lateral thoracotomy, an incision was generated on the lung with a 25G needle. Air bubbles and blood flow were detected from the defect in an immersion test with warm PBS. A UIHA solution-coated UIHA patch was used to stop the bleeding and seal the pulmonary defect. After 5-7 min for UIHA curing, the sealing effect of UIHA on lung leakage was evaluated by submerging the defect in warm PBS.

Closure of Rat Skin Incision with UIHA

Skin incision sealing capacity of UIHA was tested on rats (n=5). After anesthesia, the abdomens of the rats were shaved and disinfected with ethanol. A 2.5 cm transverse incision on rat skins was generated, and 10 μl of UIHA solution was added to the edges of incision. After 3-5 minutes for UIHA curing, the incisions were sealed effectively. In the control group, skin incisions underwent regular suturing closure (4-0 unresorbable suture).

Rat Skull Injury Sealing with UIHA

Skull injury sealing capacity of UIHA was tested on rats (n=5). After anesthesia, the tops of skulls were exposed. Craniotomy was operated to generate a square incision of 5 mm×5 mm, and 10 μl of UIHA solution was added to the defected area. In the control group, craniotomy was operated without UIHA treatment.

### Micro-Computed Tomography Angiography (CTA)

For CTA analysis, 3 days after operation on the carotid arteries, the rats were anesthetized as described before. A thoracotomy was performed to provide good exposure for intravascular contrast agent (Iohexol Injection, Yangzijiang Pharmaceutical Group, China) injection. The rats were euthanized by anesthetic dose, and a micro-CT scanner (Quantum FX, Perkin Elmer) was used to evaluate the patency of the rat carotid arteries.

### Histology and Immunohistology

UIHA adhesive and surrounding tissue were used for histological analysis. The sections were fixed with 4% paraformaldehyde/PBS at 4° C. overnight and then processed for H&E stainning. Anti-CD68, anti-CD3 (Abcam), primary antibodies with Alexa Fluor 568-conjugated (Life Technologies), secondary antibodies were applied to immunofluorescence stanning. The sections were further stained by Hochest 33342 (Invitrogen) for nuclei. The H&E-stained sections were imaged with a Leica microscope. The immunofluorescence-stained sections were imaged with a Zeiss confocal microscope.

### Swelling Ratios Study

The swelling ratios of UIHA at different weight fraction were calculated by dividing the measured weights of the samples after incubation at 37° C. in PBS by their corresponding dry weights at different times.

### In Vitro Cytocompatibility of UIHA

The cytocompatibility of UIHA at different weight fractions was examined by using endothelial cells and a live/dead assay. Briefly, endothelial cells were seeded and cultured on the surface of the UIHA for 24 hours at 37° C. and 5% CO₂. Cell viability test was performed with a live/dead viability/cytotoxicity kit for mammalian cells. An inverted fluorescent microscope (Evos FL Auto, Life Technologies) was appled to image live (green stain) and dead (red stain) cells. ImageJ software was used to calculate the cell viability by dividing the number of the live cells by total number of cells. CCK-8 assay (Sangon Biotech) test was also carried out to quantify the cell viability in accordance with the instruction provided by the manufacturer.

While the preferred embodiments of the invention have been described above, it will be recognized and understood that various modifications may be made therein, and the appended claims are intended to cover all such modifications which may fall within the spirit and scope of the invention.

### REFERENCES

[1] Yuk H, Varela C E, Nabzdyk C S, Mao X, Padera R F, Roche E T, et al. Dry double-sided tape for adhesion of wet tissues and devices. Nature. 2019; 575:169-74.
[2] Li J, Celiz A D. Yang J. Yang Q. Wamala I, Whyte W, et al. Tough adhesives for diverse wet surfaces. Science. 2017:357:378-81.
[3] An S, Jeon E J, Jeon J, Cho S-W. A serotonin-modified hyaluronic acid hydrogel for multifunctional hemostatic adhesives inspired by a platelet coagulation mediator. Materials Horizons. 2019; 6:1169-78.
[4] Annabi N, Zhang Y-N, Assmann A, Sani E S, Cheng G, Lassaletta A D, et al. Engineering a highly elastic human protein-based sealant for surgical applications. Science Translational Medicine. 2017;9: eaai7466.
[5] Fitter in-body bioelectronics. Nature Biomedical Engineering. 2019; 3:1-2.
[6] Rotenberg M Y, Tian B. Bioelectronic devices: Long-lived recordings. Nature Biomedical Engineering. 2017; 1:0048.
[7] Ji S. Wan C. Wang T. Li Q. Chen G, Wang J, et al. Water-Resistant Conformal Hybrid Electrodes for Aquatic Endurable Electrocardiogramonitoring. Adv Mater. 2020:32:e2001496.
[8] Ball P. Water as an Active Constituent in Cell Biology. Chemical Reviews. 2008:108:74-108.
[9] Cui C, Fan C, Wu Y, Xiao M, Wu T, Zhang D, et al. Water-Triggered Hyperbranched Polymer Universal Adhesives: From Strong Underwater Adhesion to Rapid Sealing Hemostasis. Advanced Materials. 2019; 31:1905761.
[10] Israelachvili J, Wennerstrom H. Role of hydration and water structure in biological and colloidal interactions. Nature. 1996; 379:219-25.
[11] Rapp M V, Maier G P, Dobbs H A, Higdon N J, Waite J H, Butler A, et al. Defining the Catechol-Cation Synergy for Enhanced Wet Adhesion to Mineral Surfaces. Journal of the American Chemical Society. 2016; 138:9013-6.
[12] Drotlef D-M, Stepien L, Kappl M, Barnes W J P, Butt H-J, del Campo A. Insights into the Adhesive Mechanisms of Tree Frogs using Artificial Mimics. Advanced Functional Materials. 2013; 23:1137-46.
[13] Lee B P, Messersmith P B, Israelachvili J N, Waite J H. Mussel-Inspired Adhesives and Coatings. Annual Review of Materials Research. 2011:41:99-132.
[14] Lee H, Lee B P, Messersmith P B. A reversible wet/dry adhesive inspired by mussels and geckos. Nature. 2007:448:338-41.
[15] Rao P. Sun T L, Chen L, Takahashi R, Shinohara G, Guo H, et al. Tough Hydrogels with Fast, Strong, and Reversible Underwater Adhesion Based on a Multiscale Design. Advanced Materials. 2018:30:1801884.
[16] Baik S, Kim D W, Park Y. Lee T-J, Ho Bhang S, Pang C. A wet-tolerant adhesive patch inspired by protuberances in suction cups of octopi. Nature. 2017:546:396-400.
[17] Dompé M, Cedano-Serrano F J, Heckert O, van den Heuvel N, van der Gucht J, Tran Y, et al. Thermoresponsive Complex Coacervate-Based Underwater Adhesive. Advanced Materials. 2019:31:1808179.
[18] Liu X, Zhang Q, Duan L, Gao G. Tough Adhesion of Nucleobase-Tackifed Gels in Diverse Solvents. Advanced Functional Materials. 2019; 29:1900450.
[19] Liu Y. Meng H, Qian Z, Fan N, Choi W, Zhao F, et al. A Moldable Nanocomposite Hydrogel Composed of a Mussel-Inspired Polymer and a Nanosilicate as a Fit-to-Shape Tissue Sealant. Angewandte Chemie International Edition. 2017:56:4224-8.
[20] Maier G P, Rapp M V, Waite J H, Israelachvili J N, Butler A. Adaptive synergy between catechol and lysine promotes wet adhesion by surface salt displacement. Science. 2015:349:628-32.
[21] Shao H, Stewart R J. Biomimetic Underwater Adhesives with Environmentally Triggered Setting Mechanisms. Advanced Materials. 2010:22:729-33.
[22] Varenberg M, Gorb S N. Hexagonal Surface Micropattern for Dry and Wet Friction. Advanced Materials. 2009:21:483-6.
[23] Zhao Q. Lee D W, Ahn B K. Seo S, Kaufman Y, Israelachvili Jacob N, et al. Underwater contact adhesion and microarchitecture in polyelectrolyte complexes actuated by solvent exchange. Nature Materials. 2016:15:407-12.
[24] Yu J, Kan Y, Rapp M, Danner E, Wei W. Das S, et al. Adaptive hydrophobic and hydrophilic interactions of mussel foot proteins with organic thin films. Proceedings of the National Academy of Sciences. 2013:110:15680-5.
[25] Utzig T, Stock P. Valtiner M. Resolving Non-Specific and Specific Adhesive Interactions of Catechols at Solid/Liquid Interfaces at the Molecular Scale. Angewandte Chemie International Edition. 2016:55:9524-8.
[26] Zhang W. Wang R, Sun Z, Zhu X, Zhao Q. Zhang T, et al. Catechol-functionalized hydrogels: biomimetic design, adhesion mechanism, and biomedical applications. Chemical Society Reviews. 2020:49:433-64.
[27] Mckinley G. Visco-Elasto-Capillary Thinning and Break-Up of Complex Fluids. Rheology Reviews. 2005:3.
[28] Jeremy M Berg, John L Tymoczko, Stryer. L. Biochemistry. New York: W. H. Freeman; 2002.
[29] Rose S, Prevoteau A, Elzière P. Hourdet D, Marcellan A. Leibler L. Nanoparticle solutions as adhesives for gels and biological tissues. Nature. 2014:505:382-5.
[30] Lang N, Pereira M J, Lee Y, Friehs I, Vasilyev N V, Feins E N, et al. A Blood-Resistant Surgical Glue for Minimally Invasive Repair of Vessels and Heart Defects. Science Translational Medicine. 2014:6:218ra6-ra6.
[31] Han L, Wang M. Prieto-López L O. Deng X, Cui J. Self-Hydrophobization in a Dynamic Hydrogel for Creating Nonspecific Repeatable Underwater Adhesion. Advanced Functional Materials. 2020:30:1907064.
[32] Liu Q. Nian G, Yang C. Qu S. Suo Z. Bonding dissimilar polymer networks in various manufacturing processes. Nature Communications. 2018:9:846.
[33] Schalau G K, Bobenrieth A. Huber R O, Nartker L S, Thomas X. Silicone Adhesives in Medical Applications. 2018.
[34] Spear S L, Murphy D K, Slicton A, Walker P S. Inamed silicone breast implant core study results at 6 years. Plast Reconstr Surg. 2007:120.
[35] Spear S L, Murphy D K. Allergan Silicone Breast Implant USCCSG. Natrelle round silicone breast implants: Core Study results at 10 years. Plastic and Reconstructive Surgery. 2014:133:1354-61.
[36] Rohrich R J. Kaplan J, Dayan E. Silicone Implant Illness: Science versus Myth? Plastic and Reconstructive Surgery. 2019:144.
[37] de Buyl F. Silicone sealants and structural adhesives. International Journal of Adhesion and Adhesives. 2001:21:411-22.
[38] Jiesheng L. Shaopeng W. E D. Effect of coupling agent as integral blend additive on silicone rubber sealant. Journal of Applied Polymer Science. 2013; 128:2337-43.
[39] Yuk H. Zhang T. Lin S, Parada G A, Zhao X. Tough bonding of hydrogels to diverse non-porous surfaces. Nat Mater. 2016; 15:190-6.
[40] Zheng P. McCarthy T J. Rediscovering Silicones: Molecularly Smooth, Low Surface Energy, Unfilled, UV/Vis-Transparent, Extremely Cross-Linked, Thermally Stable, Hard, Elastic PDMS. Langmuir. 2010:26:18585-90.
[41] Svitova T. Theodoly O, Christiano S. Hill R M, Radke C J. Wetting Behavior of Silicone Oils on Solid Substrates Immersed in Aqueous Electrolyte Solutions. Langmuir. 2002; 18:6821-9.
[42] Blake T, Haynes J. Contact-angle hysteresis. Progress in surface and membrane science. 1973; 6:125-38.
[43] Berg J C. Wettability. New York (N.Y.): Marcel Dekker: 1993.
[44] Carré A, Woehl P. Spreading of Silicone Oils on Glass in Two Geometries. Langmuir. 2006:22:134-9.
[45] Mckinley G H, Tripathi A. How to extract the Newtonian viscosity from capillary breakup measurements in a filament rheometer. Journal of Rheology. 2000:44:653-70.
[46] Renardy M. A numerical study of the asymptotic evolution and breakup of Newtonian and viscoelastic jets. Journal of Non-Newtonian Fluid Mechanics. 1995:59:267-82.
[47] Packham D E. Theories of Fundamental Adhesion. In: da Silva L F M, Ochsner A. Adams R D, editors. Handbook of Adhesion Technology. Cham: Springer International Publishing; 2017. p. 1-31.
[48] Gent A N, Liu G L, Mazurek M. Experimental study of molecular entanglement in polymer networks. Journal of Polymer Science Part B: Polymer Physics. 1994:32:271-9.
[49] Mojsiewicz-Pieńkowska K. Size exclusion chromatography with evaporative light scattering detection as a method for speciation analysis of polydimethylsiloxanes. III. Identification and determination of dimeticone and simeticone in pharmaceutical formulations. Journal of Pharmaceutical and Biomedical Analysis. 2012:58:200-7.
[50] Shirzaei Sani E, Kheirkhah A. Rana D, Sun Z, Foulsham W. Sheikhi A, et al. Sutureless repair of corneal injuries using naturally derived bioadhesive hydrogels. Science Advances. 2019;5: eaav1281.
[51] Pandey N, Hakamivala A, Xu C. Hariharan P, Radionov B, Huang Z, et al. Biodegradable Nanoparticles Enhanced Adhesiveness of Mussel-Like Hydrogels at Tissue Interface. Advanced Healthcare Materials. 2018:7:1701069.
[52] Zhou J. Defante A P, Lin F, Xu Y, Yu J, Gao Y, et al. Adhesion Properties of Catechol-Based Biodegradable Amino Acid-Based Poly (ester urea) Copolymers Inspired from Mussel Proteins. Biomacromolecules. 2015; 16:266-74.
[53] Chung H, Grubbs R H. Rapidly Cross-Linkable DOPA Containing Terpolymer Adhesives and PEG-Based Cross-Linkers for Biomedical Applications. Macromolecules. 2012:45:9666-73.
[54] Zhou J, Bhagat V. Becker M L. Poly (ester urea)-Based Adhesives: Improved Deployment and Adhesion by Incorporation of Poly (propylene glycol) Segments. ACS Applied Materials & Interfaces. 2016:8:33423-9.
[55] Jeon E Y, Hwang B H, Yang Y J, Kim B J, Choi B-H, Jung G Y, et al. Rapidly light-activated surgical protein glue inspired by mussel adhesion and insect structural crosslinking. Biomaterials. 2015; 67:11-9.
[56] Inoue A, Yuk H, Lu B, Zhao X. Strong adhesion of wet conducting polymers on diverse substrates. Science Advances. 2020;6: eaay5394.

1. A hydrophobic adhesive comprising:
a) about 25 wt % to about 75 wt % crosslinker;
b) about 25 wt % to about 75 wt % entanglement fluid; and
c) about 0.05 wt % to about 0.3 wt % hydrosilylation agent.

2. The hydrophobic adhesive according to claim 1 comprising:
a) about 50 wt % to about 75 wt % crosslinker;
b) about 25 wt % to about 50 wt % entanglement fluid; and
c) about 0.05 wt % to about 0.15 wt % hydrosilylation agent.

3. The hydrophobic adhesive according to claim 1 comprising:
a) about 75 wt % crosslinker;
b) about 25 wt % entanglement fluid; and
c) about 0.1 wt % hydrosilylation agent.

4. The hydrophobic adhesive according to claim 1 formulated as a spray, as a gel, for syringe application or as a patch.

5. The hydrophobic adhesive according to claim 1 wherein the crosslinker is PDMS or a modified PDMS.

6. The hydrophobic adhesive according to claim 5 wherein the modified PDMS is functionalized PDMS or curable PDMS.

7. The hydrophobic adhesive according to claim 6 wherein the functionalized PDMS contains at least one functional group selected from vinyl groups, methacrylate, acrylate, azide and alkyne.

8. The hydrophobic adhesive according to claim 6 wherein the curable PDMS is selected from platinum catalyzed PDMS, UV-curable PDMS, peroxide catalyzed PDMS, and PDMS crosslinked through click chemistry.

9. The hydrophobic adhesive according to claim 1 wherein the entanglement fluid is a silicone with a viscosity above 10000 cS.

10. The hydrophobic adhesive according to claim 9 wherein the entanglement fluid is a silicone with a viscosity of at least 500000 cS.

11. The hydrophobic adhesive according to claim 1 wherein the entanglement fluid is a silicone with a molecular weight greater than 60000 Da.

12. The hydrophobic adhesive according to claim 1 wherein the entanglement fluid is a silicone with a molecular weight between 60000 Da to 500,000 Da.

13. The hydrophobic adhesive according to claim 1 wherein the hydrosilylation agent is a silane.

14. The hydrophobic adhesive according to claim 13 wherein the silane is vinyltrimethoxysilane, vinyltriethoxysilane, 3-(trimethoxysilyl) propyl methacrylate, allyltrimethoxysilane, or (3-mercaptopropyl) trimethoxysilane.

15. The hydrophobic adhesive according to claim 13 wherein the silane is vinyltrimethoxysilane or vinyltriethoxysilane.
